(19) United States
(12) Patent Application Publication
(10) Pub. No.: US 2009/0149435 A1
(43) Pub. Date: Jun. 11, 2009

(54) PROCESS FOR MAKING A PHARMACEUTICAL COMPOSITION

(76) Inventors: Zhengjun Wang, Irvine, CA (US); Thomas M. McGrath, Claremont, CA (US); Toan N. Ha, Irvine, CA (US); Sam W. Lam, Oceanside, CA (US); Bruce A. Firestone, Irvine, CA (US); Robert T. Lyons, Laguna Hills, CA (US); James N. Chang, Newport Beach, CA (US); John T. Trogden, Anaheim, CA (US)

Correspondence Address: ALLERGAN, INC., 2525 DUPONT DRIVE, T2-7H, IRVINE, CA, 92612-1599, US

(21) Appl. No.: 11/952,927

(22) Filed: Dec. 7, 2007

(51) Int. Cl. A61K 31/573

(52) U.S. Cl. 514/180

(57) ABSTRACT

Process for making a sterile, injectable pharmaceutical composition by combining triamcinolone, a buffer and a high viscosity hyaluronate.

### RELATED PATENT APPLICATIONS

This application is related to application Ser. No. 11/828,561, filed Jul. 26, 2007, which is a continuation in part of application Ser. No. 11/741,366, filed Apr. 27, 2007, which is a continuation in part of application Ser. No. 11/354,415, filed Feb. 14, 2006, which is a continuation in part of application Ser. No. 10/966,764, filed Oct. 14, 2004, which application claims the benefit of provisional patent application Ser. No. 60/519,237, filed Nov. 12, 2003 and provisional patent application Ser. No. 60/530,062, filed Dec. 16, 2003, all of which applications are hereby incorporated herein by reference in their entireties.

### BACKGROUND

The present invention relates to processes for making corticosteroid compositions and methods for treating and/or preventing ocular conditions, such as anterior ocular conditions and posterior ocular conditions, as well as for treating various articular pathologies. In particular the present invention relates to extended release and sustained release triamcinolone compositions, including injectable implants, for treating posterior ocular conditions, as well as for treating joint inflammation and/or joint pain.

A pharmaceutical composition (synonymously a composition) is a formulation which contains at least one active ingredient (for example a corticosteroid such as a triamcinolone) as well as, for example, one or more excipients, buffers, carriers, stabilizers, preservatives and/or bulking agents, and is suitable for administration to a patient to achieve a desired effect or result. The pharmaceutical compositions disclosed herein can have diagnostic, therapeutic, cosmetic and/or research utility in various species, such as for example in human patients or subjects.

An ocular condition can include a disease, aliment or condition which affects or involves the eye or one of the parts or regions of the eye. Broadly speaking the eye includes the eyeball and the tissues and fluids which constitute the eyeball, the periocular muscles (such as the oblique and rectus muscles) and the portion of the optic nerve which is within or adjacent to the eyeball. An anterior ocular condition is a disease, ailment or condition which affects or which involves an anterior (i.e. front of the eye) ocular region or site, such as a periocular muscle, an eye lid or an eye ball tissue or fluid which is located anterior to the posterior wall of the lens capsule or ciliary muscles. Thus, an anterior ocular condition primarily affects or involves, the conjunctiva, the cornea, the conjunctiva, the anterior chamber, the iris, the posterior chamber (behind the retina but in front of the posterior wall of the lens capsule), the lens or the lens capsule and blood vessels and nerve which vascularize or innervate an anterior ocular region or site. A posterior ocular (also referred to herein synonymously as a “posterior segment”) condition is a disease, ailment or condition which primarily affects or involves a posterior ocular region or site such as choroid or sclera (in a position posterior to a plane through the posterior wall of the lens capsule), vitreous, vitreous chamber, retina, optic nerve (i.e. the optic disc), and blood vessels and nerves which vascularize or innervate a posterior ocular (or posterior segment) region or site.

Thus, a posterior ocular condition can include a disease, ailment or condition, such as for example, macular degeneration (such as non-exudative age related macular degeneration and exudative age related macular degeneration); choroidal neovascularization; acute macular neuroretinopathy; macular edema (such as cystoid macular edema and diabetic macular edema); Behcet's disease, retinal disorders, diabetic retinopathy (including proliferative diabetic retinopathy); retinal arterial occlusive disease; central retinal vein occlusion; uveitis (including intermediate and anterior uveitis); retinal detachment; ocular trauma which affects a posterior ocular site or location; a posterior ocular condition caused by or influenced by an ocular laser treatment; posterior ocular conditions caused by or influenced by a photodynamic therapy; photocoagulation; radiation retinopathy; epiretinal membrane disorders; branch retinal vein occlusion; anterior ischemic optic neuropathy; non-retinopathy diabetic retinal dysfunction, retinitis pigmentosa and glaucoma. Glaucoma can be considered a posterior ocular condition because a therapeutic goal can be to prevent the loss of or reduce the occurrence of loss of vision due to damage to or loss of retinal cells or optic nerve cells (i.e. neuroprotection).

An anterior ocular condition can include a disease, ailment or condition, such as for example, aphakia; pseudophakia; astigmatism; blepharospasm; cataract; conjunctival diseases; conjunctivitis; corneal diseases; corneal ulcer; dry eye syndromes; eyelid diseases; lacrimal apparatus diseases; lacrimal duct obstruction; myopia; presbyopia; pupil disorders; refractive disorders and strabismus. Glaucoma can also be considered to be an anterior ocular condition because a clinical goal of glaucoma treatment can be to reduce a hypertension of aqueous fluid in the anterior chamber of the eye (i.e. reduce intraocular pressure).

Macular edema is a major cause of visual loss in patients with diabetes, central retinal vein occlusion (CRVO) and branch retinal vein occlusion (BRVO). Although laser photocoagulation can reduce further vision loss in patients with diabetic macular edema (DME), vision that has already been decreased by macular edema usually does not improve by use of laser photocoagulation. Currently, there is no FDA (U.S. Food and Drug Administration) approved treatment for macular edema associated with CRVO. For macular edema associated with BRVO, grid laser photocoagulation may be an effective treatment for some patients.

Diabetic macular edema results from abnormal leakage of macromolecules, such as lipoproteins, from retinal capillaries into the extravascular space followed by an oncotic influx of water into the extravascular space. Abnormalities in the retinal pigment epithelium may also cause or contribute to diabetic macular edema. These abnormalities can allow increased fluid from the choriocapillaries to enter the retina or they may decrease the normal efflux of fluid from the retina to the choriocapillaries. The mechanism of breakdown of the blood-retina barrier at the level of the retinal capillaries and the retinal pigment epithelium may be due to changes to tight junction proteins such as occludin. Antcliff R., et al Marshall J., The pathogenesis of edema in diabetic maculopathy, Semin Ophthalmol 1999; 14:223-232.

Macular edema from venous occlusive disease can result from thrombus formation at the lamina cribrosa or at an arteriovenous crossing. These changes can result in an increase in retinal capillary permeability and accompanying retinal edema. The increase in retinal capillary permeability and subsequent retinal edema can ensue from of a breakdown of the blood retina barrier mediated in part by vascular endothelial growth factor (VEGF), a 45 kD glycoprotein, as it is known that VEGF can increase vascular permeability. VEGF may regulate vessel permeability by increasing phosphorylation of tight junction proteins such as occludin and zonula occluden. Similarly, in human non-ocular disease states such as ascites, VEGF has been characterized as a potent vascular permeability factor (VPF).

The normal human retina contains little or no VEGF; however, hypoxia causes upregulation of VEGF production. Disease states characterized by hypoxia-induced VEGF upregulation include CRVO and BRVO. This hypoxia induced upregulation of VEGF can be inhibited pharmacologically. Pe'er J. et al., Vascular endothelial growth factor upregulation in human central retinal vein occlusion, Ophthalmology 1998; 105:412-416. It has been demonstrated that anti-VEGF antibodies can inhibit VEGF driven capillary endothelial cell proliferation. Thus, attenuation of the effects of VEGF introduces a rationale for treatment of macular edema from venous occlusive disease.

Corticosteroids, a class of substances with anti-inflammatory properties, have been demonstrated to inhibit the expression of the VEGF gene. Nauck M. et al., Induction of vascular endothelial growth factor by platelet-activating factor and platelet-derived growth factor is downregulated by corticosteroids, Am J Resp Cell Mol Biol 1997; 16:398-406. Additionally, corticosteroids can downregulate the induction of VEGF by the pro-inflammatory mediators PDGF and platelet-activating factor (PAF) in a time and dose-dependent manner. Nauck M. et al., Corticosteroids inhibit the expression of the vascular endothelial growth factor gene in human vascular smooth muscle cells, Euro J Pharmacol 1998; 341:309-315. Thus, corticosteroids can to downregulate VEGF production and reduce breakdown of the blood-retinal barrier. Certain steroids can also have antiangiogenic properties possibly due to attenuation of the effects of VEGF. It should be noted that although certain corticosteroids can apparently down regulate VEGF production there are a number of other physiological mechanisms by is which corticosteroids can effect the pathogenesis of an ocular condition, such as macular edema.

Triamcinolone

Triamcinolone is a corticosteroid and it has been reported that a saline suspension of triamcinoline (1 mg triamcinolone acetonide in 0.1 ml saline) is non-toxic upon intravitreal injection. McCuen B. et al., The lack of toxicity of intravitreally administered triamcinolone acetonide, Am J Ophthalmol 1981; 91:785-788. Intravitreal triamcinolone has been used to treat proliferative vitreoretinopathy (Jonas J. et al., Intravitreal injection of crystalline cortisone as adjunctive treatment of proliferative vitreoretinopathy, Br J Ophthalmol 2000; 84:1064-1067), as well as choroidal neovascularization (Challa J. et al., Exudative macular degeneration and intravitreal triamcinolone: 18 month follow up, Aust N Z J Ophthalmol 1998; 26:277-281; Penfold P. et al., Exudative macular degeneration and intravitreal triamcinolone: A pilot study, Aust N Z J Ophthalmol 1995; 23:293-298, and; Danis R. et al., Intravitreal triamcinolone acetonide in exudative age-related macular degeneration, Retina 2000; 20:244-250).

Additionally, European patent application 244 178 A2 (Keller) discloses intravitreal injection of an aqueous solution of dexamethasone and a hyaluronic acid, and a topical triamcinolone suspension for ear treatment is discussed in Chang H. et al., Development of a topical suspension containing three active ingredient, Drug Dev and Ind Pharm, 28(1), 29-39 (2002). Einmahl S. et al, Evaluation of a novel biomaterial in the suprachoroidal space of the rabbit eye, Invest Ophthal & Vis Sci 43(5); 1533-1539 (2002) discusses injection of a poly(ortho ester) into the suprachoroidal space, and Einmahl S. et al, Therapeutic applications of viscous and injectable poly(ortho esters), Adv Drug Del Rev 53 (2001) 45-73, discloses that a poly ortho ester polymer containing fluorouracil markedly degrades five days after intravitreal administration. See also U.S. Pat. No. 5,770,589 (Billson) which discusses intravitreal injection of a corticosteroid, such as triamcinolone acetonide. U.S. Pat. No, 5,209,926 (Babcock) discusses ophthalmic use of various amino substituted steroids.

Known formulations of triamcinolone clear (diffuses out of and/or is removed by one or more active transport mechanisms) from the vitreous within at most about 90 days, although it has been speculated that with a known formulation (Kenalog) the triamcinolone may be detectable in the vitreous for no more than four months after intravitreal injection. Thus, McCuen B. et al. (1981) supra at page 786 noted that after three months no triamcinolone was visible in any treated eyes. Others have reported that the triamcinolone present in a saline or other aqueous suspension or solution is upon intravitreal administration cleared from the vitreous in about 21-41 days; using opthalmoscopic and spectrophotometric detection means to determine disappearance of the injected triamcinolone, in non-vitrectomized rabbit eyes the average clearance rate of intravitreally triamcinolone (0.5 mg) was 41 days, while in eyes having undergone vitrectomy or combination vitrectomy and lensectomy the average clearance rate was 17 days and 7 days, respectively. Schindler R. et al., The clearance of intravitreal triamcinolone acetonide, Am J Ophthalmol 1982; 93:415-417. Using high-performance liquid chromatography (HPLC) complete clearance of intravitreally injected triamcinolone (0.4 mg) in 24 rabbit eyes was observed by 21 days. Scholes G. et al., Clearance of triamcinolone from vitreous, Arch Ophthalmol 1985; 103:1567-1569.). Such rapid clearance from the vitreous can necessitate frequent re-administration (re-dosing) in order to effectively treat an ocular condition.

A triamcinolone pharmaceutical composition available under the trade name Kenalog® (Bristol-Myers-Squibb, Princeton N.J.) has been widely used off-label to treat various ocular conditions, including by intravitreal administration. Significantly, Kenalog® is approved by the U.S. Food and Drug Administration only for intramuscular or intrabursal use, but not for the treatment of any ocular conditions. Each milliliter (ml) of Kenalog® 40 composition comprises 40 milligrams (mg) of triamcinolone acetonide, sodium chloride as a tonicity agent, 10 mg (0.99%) benzyl alcohol as a preservative, 7.5 mg (0.75%) of carboxymethylcellulose sodium and 0.4 mg (0.04%) of polysorbate 80 as resuspension aids.

It has been reported that Kenalog has a 15 day half life in the vitreous with an effect on central macular thickness being observed for up to 140 days after intravitreal injection of the Kenalog. Aubren, F. et al., Pharmacokinetic-Pharmacodynamic modeling of the effect of Triamcinolone Acetonide on Central Macular Thickness in Patients with Diabetic Macular Edema, Inv Ophth & Vis Sci, 45(10); 3435-3441: October 2004. It has also been reported that triamcinolone can be detected in the vitreous up to 93 days after a single intravitreal injection of Kenalog (Beer P. et al., Intraocular concentration and pharmacokinetics of triamcinolone acetonide after a single intravitreal injection, Opthal 110(4); 681-686: April 2003), with the triamcinolone estimated to be potentially detectable in the vitreous for about 4 months. Inoue M. et al., Vitreous concentrations of triamcinolone acetonide in human eyes after intravitreal or subtenon injection, Am J Opth 138(6); 1046-1048: 2004.

Noninfectious endophthalmitis have been reported upon intravitreal Kenalog® injection, possibly related to the preservative, excipients and/or resuspension aids present in Kenalog® (Roth D. et al., Noninfectious endophthalmitis associated with intravitreal triamcinolone injection, Arch Opthalmol 2003; 121: 1279-1282; Sutter F. et al., Pseudo-endophthalmitis after intravitreal injection of triamcinolone, Br J Ophthalmol 2003; 87:972-974).

Additionally, the presence of benzyl alcohol preservative and polysorbate 80 surfactant in Kenalog® can potentially damage or be toxic to sensitive ocular tissues, such as retinal cells, and for this reason clinicians routinely wash the triamcinolone acetonide precipitate (which forms when Kenalog® is left standing) several times with saline to reduce the concentration of these undesirable non-active materials from the formulation. Additionally, methods have been developed to filter out of Kenalog® and from identical formulations such as Kenacort-A the preservative, surfactant, and/or resuspension (suspending agents) aids present in these formulations. Nishimura A. et al., Isolating Triamcinolone acetonide particles for intravitreal use with a porous membrane filter, Retina, vol 23(6); 777-779 (2003). Such washing and/or filtering steps are inconvenient, time consuming, and increase the possibility of microbial or endotoxin contamination that could lead to intraocular infection and inflammation.

Significantly, the triamcinolone acetonide in Kenalog® 40 tends to rapidly separate and precipitate from the remainder of the composition. For example, if Kenalog® is left standing for as short a time as about five to ten minutes a substantial separation of a triamcinolone acetonide precipitate from the remainder of the composition occurs. Unfortunately, such rapid settling of the triamcinolone also occurs with other known saline based suspensions of triamcinolone (with or with preservatives and stabilizers). Thus, if the composition is to be injected into the eye it must first be vigorously shaken and used promptly after being so shaken in order to provide a substantially uniform suspension. A substantially uniform suspension (which is not provided by Kenalog or other saline based suspensions of triamcinolone) is required in order to provide a consistent and accurate dose upon administration of the suspension to the eye. In addition, resuspension processing requires the use of the resuspension aids noted above, at least one of which is less than totally desirable for sensitive ocular tissues. At least because of the potential risk of noninfectious endophthalmitis from use of the Kenalog® vehicle, development of a preservative-free triamcinolone formulation for intraocular use to treat an ocular condition (such as a posterior ocular condition) is desirable.

Elevated intraocular pressure, that is elevated anterior chamber intraocular pressure, depends on the comparative rates of aqueous production and aqueous drainage, primarily through the trabecular meshwork. Increased intraocular pressure occurs from a variety of mechanisms such as primary or secondary angle-closure glaucoma, primary or secondary open-angle glaucoma, or combined-mechanism glaucoma. If inadequately treated, increased intraocular pressure may result in glaucomatous optic nerve changes and loss of visual field.

Known formulations of corticosteroids administered by a topical, systemic or peribulbar route can cause an increase in anterior chamber intraocular pressure. For example, following 4 to 6 weeks of topical corticosteroid administration, 5% of subjects can show an elevation in intraocular pressure of >16 mmHg and 30% of subjects may show an elevation of 6 to 15 mmHg (Armaly M., Statistical attributes of the steroid hypertensive response in the clinically normal eye, Invest Ophthalmol Vis Sci 1965; 4:187-197; Becker B,. Intraocular pressure response to topical corticosteroid, Invest Ophthalmol Vis Sci 1965; 4:198-205). Additionally, intravitreal administration of known formulations of a corticosteroid, such as triamcinolone can also result in increased intraocular pressure (Martidis A. et al., Intravitreal triamcinolone for refractory diabetic macular edema, Ophthalmology 2002; 109:920-927; Jonas J. et al., Intravitreal injection of triamcinolone for diffuse diabetic macular edema, Arch Ophthalmol 2003; 121:57-61), possibly due to the burst or high release rates of triamcinolone from the known formulations.

As well as causing an increase in intraocular pressure, corticosteroids can also cause an increase in cataract formation. Corticosteroid-induced cataracts typically show an axial, posterior subcapsular opacity, which gradually increases in size. Nuclear sclerosis is not a typical lens change from corticosteroids. Topical, systemic and peribulbar corticosteroid administration have all been associated with an increased risk of cataract formation (Butcher J. et al., Bilateral cataracts and glaucoma induced by long term use of steroid eye drops. BMJ 1994; 309-343).

The intravitreal administration of known triamcinolone formulations can therefore also be expected to be associated with an increased risk of both elevated intraocular is pressure and cataract formation.

A further adverse effect from ocular corticosteroid administration can be inflammation. Endophthalmitis is a type of intraocular inflammation that can be due to infection with pathogens such as bacteria of fungi or can be noninfectious. Clinical features include lid edema, conjunctival injection, corneal edema, anterior chamber and vitreous inflammation and hypotonia. Infectious endophthalmitis can occur following an intraocular procedure (i.e. cataract surgery, vitrectomy surgery, intravitreal injection), as a result of systemic infection, as a result of trauma, or occur as a late feature of conjunctival filtering blebs.

The most common dose of triamcinolone used to treat eyes with macular edema associated with diabetes, CRVO or BRVO is 4 mg (Martidis A. et al., Intravitreal triamcinolone for refractory diabetic macular edema, Ophthalmology 2002; 109:920-927). The use of 25 mg of triamcinolone has less commonly been used to treat eyes with macular edema (Jonas J. et al., Intraocular injection of crystalline cortisone as adjunctive treatment of diabetic macular edema, Am J Ophthalmol 2001; 132:425-427).

Thus, there are significant drawbacks and deficiencies with the known triamcinolone formulations used by intravitreal administration to treat an ocular condition, including for example rapid clearance from the vitreous, elevated intraocular pressure, cataract formation, retinal toxicity, and intraocular inflammation, such as endophthalmitis.

Hence, a sterile, preservative-free, sustained release triamcinolone preparation is desirable. Additionally, because corticosteroids have known ocular toxicities (as manifested in the occurrence or development of for example elevated IOP, glaucoma and cataract) it is desirable to have a triamcinolone formulation for intraocular (i.e. intravitreal) use which does not result in an increased incidence of elevated IOP, glaucoma, cataract formation and/or intraocular inflammation, or which has, subsequent to intraocular administration of a triamcinolone formulation, a reduced incidence of elevated IOP, glaucoma, cataract formation and/or intraocular inflammation as compared to currently used or known intraocular (i.e. intravitreal) use triamcinolone.

### DRAWINGS

FIG. 1 is a flow chart which diagrammatically illustrates a one vessel manufacturing process for making the triamcinolone formulations of Examples 1 and 2.

FIG. 2 is a flow chart which diagrammatically illustrates a two vessel manufacturing (compounding) process for making the triamcinolone formulations of Examples 1 and 2.

### SUMMARY

The present invention provides a process for making a sterile, preservative-free, sustained release triamcinolone formulations for treating ocular conditions with the desirable characteristics of low ocular toxicities, as manifested in the low or nominal occurrence or development of an elevated IOP, glaucoma, cataract and/or intraocular inflammation.

Definitions

As used herein, the words or terms set forth below have the following definitions.

“About” means that the item, parameter or term so qualified encompasses a range of plus or minus ten percent above and below the value of the stated item, parameter or term.

“Administration”, or “to administer” means the step of giving (i.e. administering) a pharmaceutical composition to a subject. The pharmaceutical compositions disclosed herein can be “locally administered”, that is administered at or in the vicinity of the site at which a therapeutic result or outcome is desired. For example to treat an ocular condition (such as for example a macular edema, uveitis or macular degeneration) intravitreal injection or implantation of a sustained release device such as active agent containing polymeric implant can be carried out. “Sustained release” means release of an active agent (such as a triamcinolone) over a period of about seven days or more, while “extended release” means release of an active agent over a period of time of less than about seven days.

“Entirely free (i.e. “consisting of” terminology) means that within the detection range of the instrument or process being used, the substance cannot be detected or its presence cannot be confirmed.

“Essentially free” (or “consisting essentially of”) means that only trace amounts of the substance can be detected.

“Pharmaceutical composition” means a formulation in which an active ingredient (the active agent) can be a steroid, such as a corticosteroid, such as a triamcinolone. The word “formulation” means that there is at least one additional ingredient in the pharmaceutical composition besides the steroid active ingredient. A pharmaceutical composition is therefore a formulation which is suitable for diagnostic or therapeutic administration (i.e. by intraocular injection or by insertion of a depot or implant) to a subject, such as a human patient.

“Substantially free” means present at a level of less than one percent by weight of the pharmaceutical composition.

All the viscosity values set forth herein were determined at 25° C. (unless another temperature is specified). Additionally, all the viscosity values set forth herein were determined at a shear rate of about 0.1/second (unless another shear rate is specified).

The present compositions are highly suitable for intravitreal administration into the posterior segments of an eye with little or no retinal damage. The present compositions are advantageously substantially free or entirely of added preservative components, for example, contain no benzyl alcohol preservative. In addition, the present compositions advantageously require no resuspension aid or aids. Overall, the present compositions are easily and effectively injectable into the posterior segment of an eye of a human or animal and can be maintained as a substantially uniform suspension for long periods of time, for example, at least about one week or more, without resuspension processing, for example, without requiring shaking or other agitating of the composition to obtain substantial suspension uniformity. In short, the present compositions and methods provide substantial enhancements and advantages, for example, relative to the prior art Kenalog® 40 composition and methods of using such prior art composition, in the posterior segments of human or animal eyes.

In one broad aspect of the present invention, compositions useful for injection into a posterior segment of an eye of a human or animal are provided. Such compositions comprise a corticosteroid component, a viscosity inducing component, and an aqueous carrier component. The corticosteroid component is present in a therapeutically effective amount. The corticosteroid component is present in the compositions in a plurality of particles.

The present invention also includes a process for making a pharmaceutical composition by (a) mixing triamcinolone particles about 4 microns to about 8 microns in diameter with sodium chloride crystals, and about 35% to about 40% of the total volume of the water (water for injection) used to make the formulation; (b) heating the triamcinolone and sodium chloride mixture to a temperature between about 120° C. and about 140° C., thereby preparing a first part; (c) mixing a sodium phosphate and water, thereby preparing a second part; (d) dissolving sodium hyaluronate with a molecular weight between about 1.0 million Daltons and about 1.9 million Daltons in another about 35% to about 40% of the total water volume used to make the formulation, followed by sterile filtration after the dissolving; (e) lyophilization of the dissolved sodium hyaluronate; (f) reconstitution of the lyophilized, sterile sodium hyaluronate, thereby preparing a third part; and; (g) aseptically combining the first, second and third parts, thereby making a sterile, uniform triamcinolone pharmaceutical composition which is, an opaque white gel suspension suitable for intravitreal injection to treat an ocular condition. Water is added as needed (q.s.) to make the desired gel suspension which is about 80% to about 90% by weight water.

The present invention includes a process for making a pharmaceutical composition by preparing a first part comprising an aqueous suspension of particles of an anti-inflammatory steroid; sterilizing the first part; preparing a second part comprising sterile buffer solution for providing a physiological pH to the pharmaceutical composition; preparing a third part comprising sterile high viscosity carrier for the anti-inflammatory steroid; mixing together the first, second and third parts to thereby make or provide a sterile, physiological pH pharmaceutical composition with a viscosity of between about 130,000 cps and about 300,000 cps at a shear rate of about 0.1/second at about 25° C. suitable for treating an inflammatory condition.

The first part can also comprises sodium chloride and the anti-inflammatory steroid can be triamcinolone. Additionally, the sterilizing step can be carried out by heating the first part. Thus, the first part can be heated to about 115° C. to about 135° C. for between about 10 minutes and about 90 minutes.

The second part can comprises an aqueous phosphate buffer and the third part can comprise a sodium hyaluronate. Importantly, the mixing step is carried out adding part 3 to part 2, followed by addition of part 1 and the steps of preparing first part and sterilizing the first part are carried out in a first vessel and the mixing step is carried out in a second vessel. Alternately, the steps of preparing first part and sterilizing the first part and the mixing step are carried out in the same vessel.

A detailed process for making a pharmaceutical composition within the scope of our invention can have the steps of:
(a) preparing a first part comprising sodium chloride and an aqueous suspension of particles of triamcinolone;
(b) sterilizing the first part by heating the first part
(c) preparing a second part comprising sterile phosphate buffer solution for providing a physiological pH to the pharmaceutical composition;
(d) preparing a third part comprising sterile high viscosity sodium hyaluronate carrier for the triamcinolone;
(e) mixing together the first, second and third parts by adding part 3 to part 2 followed by add of part 1, to thereby provide a sterile, physiological pH pharmaceutical composition with a viscosity of between about 130,000 cps and about 300,000 cps at a shear rate of about 0.1/second at about 25° C., suitable for treating an inflammatory condition and injectable through a 27 gauge needle.

### DESCRIPTION

The present invention is based upon our discovery of processes for making triamcinolone formulations specifically designed for intravitreal injection to treat various ocular conditions, such a macula edema. Our triamcinolone formulations have numerous superior characteristics and advantages, including the following: (1) the triamcinolone present in our formulations does not rapidly settle out from or precipitate from the formulations. Importantly our formulations have a shelf life of at least two years, meaning that our formulations can be left standing for up to about two years before administration to an eye, and after two years the formulation can still provide a consistent and accurate dose of triamcinolone upon injection to the formulation to an eye; (2) our formulations are free of preservatives and resuspension aids, such as benzyl alcohol and/or a polysorbate; (3) concomitantly, our formulations have a much reduced retinal and photoreceptor toxicity; (4) as well as being sterile and preservative-free our triamcinolone formulations can provide sustained release of therapeutic amounts of the triamcinolone over multi-month periods upon intravitreal injection of such formulations. Thus, our viscous suspension triamcinolone formulations can be characterized as sustained release implants; (5) intravitreal administration of our triamcinolone formulations is not associated with an increased incidence of adverse events such as elevated intra ocular pressure, glaucoma, cataract and/an intraocular inflammation; (6) intravitreal administration of our triamcinolone formulations is not associated with an increased incidence of adverse events such elevated intra ocular pressure, glaucoma, cataract and/an intraocular inflammation as compared to currently used or known intraocular (i.e. intravitreal) use triamcinolone formulations; (7) our formulations permit triamcinolone particles (crystals) to be released (as they solubilize in the viscous fluid of the posterior chamber) from a discrete unitary location, thereby avoiding the plume effect (rapid dispersion) characteristic of aqueous triamcinolone formulations upon intravitreal administration; (8) avoidance of plume formation or rapid dispersion upon intravitreal administration beneficially reduces visual field obscuration; (9) the sustained release characteristic of our formulations reduces the need for intravitreal administration of large drug quantities to achieve a desired therapeutic effect; (10) upon intravitreal administration, the triamcinolone present in our formulations can preferentially be eliminated in posterior direction (that is through the retina and optic nerve) as opposed to elimination through an anterior route. This can result in superior treatment of a retinal disease with for example reduced ocular hypertension.

Advantage (1) above can be provided by formulating the triamcinolone as a viscous, gel suspension, as opposed to formulating it as an aqueous or saline based formulation. Additionally, advantages (4) and (8) above can be provided by particular characteristics of our formulations, such as suspension of the triamcinolone in one or more particular high molecular weight hydrogel polymers which permit sustained release of the triamcinolone from a biocompatible, biodegradable polymeric matrix, and other implant-like characteristics to the formulations, including substantially zero-order in vivo (i.e. intravitreal) release kinetics.

The pluming effect occurs when a saline suspension of a triamcinolone (such as Kenalog) is injected into the vitreous. Pluming prevents visualization of the back of the eye (i.e. the retina is obscured) and also reduces the patient's visual field (reduced vision).

Generally, the present invention provides compositions useful for placement, preferably by injection, into a posterior segment of an eye of a human or animal. Such compositions in the posterior, e.g., vitreous, of the eye are therapeutically effective against one or more conditions and/or diseases of the posterior of the eye, and/or one or more symptoms of such conditions and/or diseases of the posterior of the eye.

It is important to note that while preferably the compositions disclosed herein are preferably administered by intravitreal injection to treat a posterior ocular condition, our compositions (i.e. those of Examples 1 and 2) can also be administered (as by injection) by other routes, such as for example subconjuctival, sub-tenon, periocular, retrobulbar, suprachoroidal, and/or intrascleral to effectively treat an ocular condition. Additionally, a sutured on refillable dome can be placed over the administration site to prevent or to reduce wash out, leaching and/or diffusion of the active agent in a non-preferred direction.

Compositions within the scope of our invention can comprise a corticosteroid component; a viscosity inducing component; and an aqueous carrier component. The compositions are advantageously ophthalmically acceptable. One of the important advantages of the present compositions is that they are more compatible with or friendly to the tissues in the posterior segment of the eye, for example, the retina of the eye, relative to compositions previously proposed for intravitreal injection into a posterior segment of an eye, for example, a composition sold under the trademark Kenalog®-40. In particular, the present compositions advantageously are substantially free of added preservative components or include effective preservative components which are more compatible with or friendly to the posterior segment, e.g., retina, of the eye relative to benzyl alcohol, which is included in the Kenalog®-40 composition as a preservative.

In addition, the present compositions preferably include no added resuspension component, such as polysorbate-80, which is included in the Kenalog®-40 composition. Many of the other features of the present compositions, as described elsewhere herein, also render the present compositions more compatible with or friendly to the posterior segments of the eyes into which the compositions are placed relative to prior art compositions, such as Kenalog®-40.

As noted above, the present compositions include a corticosteroid component. Such corticosteroid component is present in the compositions in a therapeutically effective amount, that is in an amount effective in providing a desired therapeutic effect in the eye into which the composition is placed. The corticosteroid component is present in the composition in a plurality of particles. Any suitable corticosteroid component may be employed in according to the present invention. Such corticosteroid component advantageously has a limited solubility in water, for example, at 25° C. For example, the corticosteroid component preferably has a solubility in water at 25° C. of less than 10 mg/ml. Of course, the corticosteroid component should be ophthalmically acceptable, that is, should have substantially no significant or undue detrimental effect of the eye structures or tissues. One particularly useful characteristic of the presently useful corticosteroid components is the ability of such component to reduce inflammation in the posterior segment of the eye into which the composition is placed caused by the result of one or more diseases and/or conditions in the posterior segment of the eye.

Examples of useful corticosteroid components include, without limitation, cortisone, prednesolone, triamcinolone, triamcinolone acetonide, fluorometholone, dexamethosone, medrysone, loteprednol, derivatives thereof and mixtures thereof. As used herein, the term “derivative” refers to any substance which is sufficiently structurally similar to the material of which it is identified as a derivative so as to have substantially similar functionality or activity, for example, therapeutic effectiveness, as the material when the substance is used in place of the material. In one very useful embodiment, the corticosteroid component comprises triamcinolone acetonide.

The corticosteroid component advantageously is present in an amount of at least about 10 mg per ml of the composition. One important advantage of the present invention is the effective ability of the present compositions to include relatively large amounts or concentrations of the corticosteroid component. Thus, the corticosteroid component may be present in the present compositions in an amount in the range of about 1% or less to about 5% or about 10% or about 20% or about 30% or more (w/v) of the composition. Providing relatively high concentrations or amounts of corticosteroid component in the present compositions is beneficial in that reduced amounts (volumes for injection) of the composition may be required to be placed or injected into the posterior segment of the eye in order to provide the same amount or more corticosteroid component in the posterior segment of the eye relative to compositions, such as Kenalog®-40, which include less than 4% (w/v) of the corticosteroid component. Thus, in one very useful embodiment, the present compositions include more than about 4% (w/v), for example at least about 5% (w/v), to about 10% (w/v) or about 20% (w/v) or about 30% (w/v) of the corticosteroid component. For example, about 50 μL of our Example 1 or 2 formulation provide respectively 2 mg and 4 mg of triamcinolone. This is in contrast to other formulations (such as Kenalog 40) which require 100 μL to provide 4 mg of triamcinolone. Injection of 100 μL or more of a fluid into the vitreous can result in an excess of fluid in the vitreous with elevated intraocular pressure and leakage of the fluid from the vitreous then potentially occurring.

The viscosity inducing component is present in an effective amount in increasing, advantageously substantially increasing, the viscosity of the composition. Without wishing to limit the invention to any particular theory of operation, it is believed that increasing the viscosity of the compositions to values well in excess of the viscosity of water, for example, at least about 100 cps at a shear rate of 0.1/second, compositions which are highly effective for placement, e.g., injection, into the posterior segment of an eye of a human or animal are obtained. Along with the advantageous placement or injectability of the present compositions into the posterior segment, the relatively high viscosity of the present compositions are believed to enhance the ability of the present compositions to maintain the corticosteroid component particles in substantially uniform suspension in the compositions for prolonged periods of time, for example, for as long as 1 to 2 years, without requiring resuspension processing. The relatively high viscosity of the present compositions may also have an additional benefit of at least assisting the compositions to have the ability to have an increased amount or concentration of the corticosteroid component, as discussed elsewhere herein, for example, while maintaining such corticosteroid component in substantially uniform suspension for prolonged periods of time.

Advantageously, the present compositions have viscosities of at least about 10 cps or at least about 100 cps or at least about 1000 cps, more preferably at least about 10,000 cps and still more preferably at least about 70,000 cps or more, for example up to about 200,000 cps or about 250,000 cps, or about 300,000 cps or more, at a shear rate of 0.1/second. The present compositions not only have the relatively high viscosity as noted above but also have the ability or are structured or made up so as to be effectively placeable, e.g., injectable, into a posterior segment of an eye of a human or animal, preferably through a 27 gauge needle, or even through a 30 gauge needle.

The presently useful viscosity inducing components preferably are shear thinning components in that as the present composition containing such a shear thinning viscosity inducing component is passed or injected into the posterior segment of an eye, for example, through a narrow space, such as 27 gauge needle, under high shear conditions the viscosity of the composition is substantially reduced during such passage. After such passage, the composition regains substantially its pre-injection viscosity so as to maintain the corticosteroid component particles in suspension in the eye.

Any suitable viscosity inducing component, for example, ophthalmically acceptable viscosity inducing component, may be employed in accordance with the present invention. Many such viscosity inducing components have been proposed and/or used in ophthalmic compositions used on or in the eye. The viscosity inducing component is present in an amount effective in providing the desired viscosity to the composition. Advantageously, the viscosity inducing component is present in an amount in a range of about 0.5% or about 1.0% to about 5% or about 10% or about 20% (w/v) of the composition. The specific amount of the viscosity inducing component employed depends upon a number of factors including, for example and without limitation, the specific viscosity inducing component being employed, the molecular weight of the viscosity inducing component being employed, the viscosity desired for the present composition being produced and/or used and the like factors, such as shear thinning. The viscosity inducing component is chosen to provide at least one advantage, and preferably multiple advantages, to the present compositions, for example, in terms of each of injectability into the posterior segment of the eye, viscosity, sustainability of the corticosteroid component particles in suspension, for example, in substantially uniform suspension, for a prolonged period of time without resuspension processing, compatibility with the tissues in the posterior segment of the eye into which the composition is to be placed and the like advantages. More preferably, the selected viscosity inducing component is effective to provide two or more of the above-noted benefits, and still more preferably to provide all of the above-noted benefits.

The viscosity inducing component preferably comprises a polymeric component and/or at least one viscoelastic agent, such as those materials which are useful in ophthalmic surgical procedures.

Examples of useful viscosity inducing components include, but are not limited to, hyaluronic acid (such as a polymeric hyaluronic acid), carbomers, polyacrylic acid, cellulosic derivatives, polycarbophil, polyvinylpyrrolidone, gelatin, dextrin, polysaccharides, polyacrylamide, polyvinyl alcohol, polyvinyl acetate, derivatives thereof and mixtures and copolymers thereof.

The molecular weight of the presently useful viscosity inducing components may be in a range of about 10,000 Daltons or less to about 2 million Daltons or more. In one particularly useful embodiment, the molecular weight of the viscosity inducing component is in a range of about 100,000 Daltons or about 200,000 Daltons to about 1 million Daltons or about 1.5 million Daltons. Again, the molecular weight of the viscosity inducing component useful in accordance with the present invention, may vary over a substantial range based on the type of viscosity inducing component employed, and the desired final viscosity of the present composition in question, as is well as, possibly one or more other factors.

In one very useful embodiment, a viscosity inducing component is a polymeric hyaluronate component, for example, a metal hyaluronate component, preferably selected from alkali metal hyaluronates, alkaline earth metal hyaluronates and mixtures thereof, and still more preferably selected from sodium hyaluronates, and mixtures thereof. The molecular weight of such hyaluronate component (i.e. a polymeric hyaluronic acid) preferably is in a range of about 50,000 Daltons or about 100,000 Daltons to about 1.3 million Daltons or about 2 million Daltons. In one embodiment, the present compositions include a polymeric hyaluronate component in an amount in a range about 0.05% to about 0.5% (w/v). In a further useful embodiment, the hyaluronate component is present in an amount in a range of about 1% to about 4% (w/v) of the composition. In this latter case, the very high polymer viscosity forms a gel that slows particle sedimentation rate to the extent that often no resuspension processing is necessary over the estimated shelf life, for example, at least about 2 years, of the composition. Such a composition may be marketed in pre-filled syringes since the gel cannot be easily removed by a needle and syringe from a bulk container. Pre-filled syringes have the advantages of convenience for the injector and the safety which results from less handling.

The aqueous carrier component is advantageously ophthalmically acceptable and may include one or more conventional excipients useful in ophthalmic compositions. The present compositions preferably include a major amount of liquid water. The present compositions may be, and are preferably, sterile, for example, prior to being used in the eye.

The present compositions preferably include at least one buffer component in an amount effective to control the pH of the composition and/or at least one tonicity component in an amount effective to control the tonicity or osmolality of the compositions. More preferably, the present compositions include both a buffer component and a tonicity component.

The buffer component and tonicity component may be chosen from those which are conventional and well known in the ophthalmic art. Examples of such buffer components include, but are not limited to, acetate buffers, citrate buffers, phosphate buffers, borate buffers and the like and mixtures thereof. Phosphate buffers are particularly useful. Useful tonicity components include, but are not limited to, salts, particularly sodium chloride, potassium chloride, mannitol and other sugar alcohols, and other suitable ophthalmically acceptably tonicity component and mixtures thereof.

The amount of buffer component employed preferably is sufficient to maintain the pH of the composition in a range of about 6 to about 8, more preferably about 7 to about 7.5. The amount of tonicity component employed preferably is sufficient to provide an osmolality to the present compositions in a range of about 200 to about 400, more preferably about 250 to about 350, mOsmol/kg respectively. Advantageously, the present compositions are substantially isotonic.

The present compositions may include one or more other components in amounts effective to provide one or more useful properties and/or benefits to the present compositions. For example, although the present compositions may be substantially free of added preservative components, in other embodiments, the present compositions include effective amounts of preservative components, preferably such components which are more compatible with or friendly to the tissue in the posterior segment of the eye into which the composition is placed than benzyl alcohol. Examples of such preservative components include, without limitation, benzalkonium chloride, chlorhexidine, PHMB (polyhexamethylene biguanide), methyl and ethyl parabens, hexetidine, chlorite components, such as stabilized chlorine dioxide, metal chlorites and the like, other ophthalmically acceptable preservatives and the like and mixtures thereof. The concentration of the preservative component, if any, in the present compositions is a concentration effective to preserve the composition, and is often in a range of about 0.00001% to about 0.05% or about 0.1% (w/v) of the composition.

In addition, the present composition may include an effective amount of resuspension component effective to facilitate the suspension or resuspension of the corticosteroid component particles in the present compositions. As noted above, in certain embodiments, the present compositions are free of added resuspension components. In other embodiments of the present compositions effective amounts of resuspension components are employed, for example, to provide an added degree of insurance that the corticosteroid component particles remain in suspension, as desired and/or can be relatively easily resuspended in the present compositions, such resuspension be desired. Advantageously, the resuspension component employed in accordance with the present invention, if any, is chosen to be more compatible with or friendly to the tissue in the posterior segment of the eye into which the composition is placed than polysorbate 80.

Any suitable resuspension component may be employed in accordance with the present invention. Examples of such resuspension components include, without limitation, surfactants such as poloxanes, for example, sold under the trademark Pluronic®; tyloxapol; sarcosinates; polyethoxylated castor oils, other surfactants and the like and mixtures thereof.

One very useful class of resuspension components are those selected from vitamin derivatives. Although such materials have been previously suggested for use as surfactants in ophthalmic compositions, they have been found to be effective in the present compositions as resuspension components. Examples of useful vitamin derivatives include, without limitation, Vitamin E tocopheryl polyethylene glycol succinates, such as Vitamin E tocopheryl polyethylene glycol 1000 succinate (Vitamin E TPGS). Other useful vitamin derivatives include, again without limitation, Vitamin E tocopheryl polyethylene glycol succinamides, such as Vitamin E tocopheryl polyethylene glycol 1000 succinamide (Vitamin E TPGSA) wherein the ester bond between polyethylene glycol and succinic acid is replaced by an amide group.

The presently useful resuspension components are present, if at all, in the compositions in accordance with the present invention in an amount effective to facilitate suspending the particles in the present compositions, for example, during manufacture of the compositions or thereafter. The specific amount of resuspension component employed may vary over a wide range depending, for example, on the specific resuspension component being employed, the specific composition in which the resuspension component is being employed and the like factors. Suitable concentrations of the resuspension component, if any, in the present compositions are often in a range of about 0.01% to about 5%, for example, about 0.02% or about 0.05% to about 1.0% (w/v) of the composition.

The availability of minimally soluble corticosteroid components, such as triamcinolone acetonide, to intraocular tissues may be limited by the dissolution rate for these substances. Slow dissolution is both good and bad for the patient. On the one hand, after a single intravitreal injection of the present composition, the mean elimination half-life for triamcinolone acetonide is advantageously quite long, for example, about 19 days in nonvitrectonized patients and measurable drug levels are detected for up to about 3 months. On the other hand, therapeutic drug levels in the vitreous compartment of the eye may not be achieved for about 1 to about 3 days, due to the slow dissolution rate of the corticosteroid component particles.

In one embodiment of the present invention, an effective amount of a solubilizing component is provided in the composition to solubilize a minor amount, that is less is than 50%, for example in a range of 1% or about 5% to about 10% or about 20% of the corticosteroid component. For example, the inclusion of a cyclodextrin component, such as β-cyclodextrin, sulfo-butylether β-cyclodextrin (SBE), other cyclodextrins and the like and mixtures thereof, at about 0.5 to about 5.0% (w/v) solubilizes about 1 to about 10% of the initial dose of triamcinolone acetonide. This presolubilized fraction provides a readily bioavailable loading dose, thereby avoiding any delay time in therapeutic effectiveness.

The use of such a solubilizing component is advantageous to provide any relatively quick release of the corticosteroid component into the eye for therapeutic effectiveness. Such solubilizing component, of course, should be ophthalmically acceptable or at least sufficiently compatible with the posterior segment of the eye into which the composition is placed to avoid undue damage to the tissue in such posterior segment.

The pharmacokinetics of the corticosteroid component, for example, triamcinolone acetonide, following intravitreal administration may involve both the rate of drug dissolution and the rate of drug efflux via the anterior route. For example, following a single intravitreal injection of a composition containing 4% (w/v) of triamcinolone acetonide, triamcinolone acetonide concentration peaks (monitored in aqueous humor) after several days at thousands of nanograms per mL. This peak (Cₘₐₓ) is followed by a rapid decrease lasting about 200 hours, and ends in a slow elimination phase with a half-life of about 19 days. Patients typically require repeat dosing, for example about every three months.

In one embodiment of the present invention, the compositions further contain sustained release components, for example, polymers (in the form for example of gels and microspheres), such as poly (D,L,-lactide) or poly(D,L-lactide co-glycolide), in amounts effective to reduce local diffusion rates and/or corticosteroid particle dissolution rates. The result is a flatter elimination rate profile with a lower Cₘₐₓ and a more prolonged therapeutic window, thereby extending the time between required injections for many patients.

Any suitable, preferably conditionally acceptable, release component may be employed. Useful examples are set forth above. The sustained release component is preferably biodegradable or bioabsorbable in the eye so that no residue remains over the long term. The amount of the delayed release component included may very over a relatively wide range depending, for example, on the specific sustained release component is being employed, the specific release profile desired and the like factors. Typical amounts of delayed release components, if any, included in the present compositions are in a range of about 0.05 to 0.1 to about 0.5 or about 1 or more percent (w/v) (weight of the ingredient in the total volume of the composition) of the composition.

The present compositions can be prepared using suitable blending/processing techniques or techniques, for example, one or more conventional blending techniques. The preparation processing should be chosen to provide the present compositions in forms which are useful for placement or injection into the posterior segments of eyes of humans or animals. In one useful embodiment a concentration corticosteroid component dispersion is made by combining the corticosteroid component with water, and the excipient (other than the viscosity inducing component) to be included in the final composition. The ingredients are mixed to disperse the corticosteroid component and then autoclaved. Alternatively, the steroid powder may be γ-irradiated before addition to the sterile carrier. The viscosity inducing component may be purchased sterile or sterilized by conventional processing, for example, by filtering a dilute solution followed by lyophylization to yield a sterile powder. The sterile viscosity inducing component is combined with water to make an aqueous concentrate. Under aseptic conditions, the concentrated corticosteroid component dispersion can be blended or mixed and added or combined as a slurry to the viscosity inducing component concentrate. Water is added in a quantity sufficient (q.s.) to provide the desired composition and the composition is mixed until homogenous.

Methods of using the present composition are provided and are included within the scope of the present invention. In general, such methods comprise administering a composition in accordance with the present invention to a posterior segment of an eye of a human or animal, thereby obtaining a desired therapeutic effect. The administering step advantageously comprises at least one of intravitreal injecting, subconjunctival injecting, sub-tenon injecting, retrobulbar injecting, suprachoroidal injecting and the like. A syringe apparatus including an appropriately sized needle, for example, a 27 gauge needle or a 30 gauge needle, can be effectively used to inject the composition with the posterior segment of an eye of a human or animal.

Ocular conditions which can be treated or addressed in accordance with the present invention include, without limitation, the following:

Maculopathies/retinal degeneration: macular degeneration, including age related macular degeneration (ARMD), such as non-exudative age related macular degeneration and exudative age related macular degeneration, choroidal neovascularization, retinopathy, including diabetic retinopathy, acute and chronic macular neuroretinopathy, central serous chorioretinopathy, and macular edema, including cystoid macular edema, and diabetic macular edema. veitis/retinitis/choroiditis: acute multifocal placoid pigment epitheliopathy, Behcet's disease, birdshot retinochoroidopathy, infectious (syphilis, lyme, tuberculosis, toxoplasmosis), uveitis, including intermediate uveitis (pars planitis) and anterior uveitis, multifocal choroiditis, multiple evanescent white dot syndrome (MEWDS), ocular sarcoidosis, posterior scleritis, serpignous choroiditis, subretinal fibrosis, uveitis syndrome, and Vogt-Koyanagi-Harada syndrome. Vascular diseases/exudative diseases: retinal arterial occlusive disease, central retinal vein occlusion, disseminated intravascular coagulopathy, branch retinal vein occlusion, hypertensive fundus changes, ocular ischemic syndrome, retinal arterial microaneurysms, Coat's disease, parafoveal telangiectasis, hemi-retinal vein occlusion, papillophlebitis, central retinal artery occlusion, branch retinal artery occlusion, carotid artery disease (CAD), frosted branch angitis, sickle cell retinopathy and other hemoglobinopathies, angioid streaks, familial exudative vitreoretinopathy, Eales disease. Traumatic/surgical: sympathetic ophthalmia, uveitic retinal disease, retinal detachment, trauma, laser, PDT, photocoagulation, hypoperfusion during surgery, radiation retinopathy, bone marrow transplant retinopathy. Proliferative disorders: proliferative vitreal retinopathy and epiretinal membranes, proliferative diabetic retinopathy. Infectious disorders: ocular histoplasmosis, ocular toxocariasis, presumed ocular histoplasmosis syndrome (POHS), endophthalmitis, toxoplasmosis, retinal diseases associated with HIV infection, choroidal disease associated with HIV infection, uveitic disease associated with HIV Infection, viral retinitis, acute retinal necrosis, progressive outer retinal necrosis, fungal retinal diseases, ocular syphilis, ocular tuberculosis, diffuse unilateral subacute neuroretinitis, and myiasis. Genetic disorders: retinitis pigmentosa, systemic disorders with associated retinal dystrophies, congenital stationary night blindness, cone dystrophies, Stargardt's disease and fundus flavimaculatus, Bests disease, pattern dystrophy of the retinal pigmented epithelium, X-linked retinoschisis, Sorsby's fundus dystrophy, benign concentric maculopathy, Bietti's crystalline dystrophy, pseudoxanthoma elasticum. Retinal tears/holes: retinal detachment, macular hole, giant retinal tear. Tumors: retinal disease associated with tumors, congenital hypertrophy of the RPE, posterior uveal melanoma, choroidal hemangioma, choroidal osteoma, choroidal metastasis, combined hamartoma of the retina and retinal pigmented epithelium, retinoblastoma, vasoproliferative tumors of the ocular fundus, retinal astrocytoma, intraocular lymphoid tumors. Miscellaneous: punctate inner choroidopathy, acute posterior multifocal placoid pigment epitheliopathy, myopic retinal degeneration, acute retinal pigment epithelitis and the like.

The present methods may comprise a single injection into the posterior segment of an eye or may involve repeated injections, for example over periods of time ranging from about one week or about 1 month or about 3 months to about 6 months or about 1 year or longer.

### EXAMPLES

The following non-limiting Examples are presented to exemplify aspects of the present invention.

### Examples 1 and 2

Two compositions are as follows:

TABLE 1

| Ingredient | Example 1 | Example 2 |
|---|---|---|
| Triamcinolone acetonide | 2.0% (w/v) | 8.0% (w/v) |
| Sodium hyaluronate (polymeric) | 2.5% (w/v) | 2.3% (w/v) |
| Sodium chloride | 0.63% (w/v) | 0.63% (w/v) |
| dibasic sodium phosphate, heptahydrate | 0.30% (w/v) | 0.30% (w/v) |
| Monobasic sodium phosphate, monohydrate | 0.04% (w/v) | 0.04% (w/v) |
| Water for Injection | q.s. | q.s. |
| Viscosity at shear rate 0.1/second at 25° C. | 170,000 ± | 200,000 ± |
| | 25% cps | 25% cps |

These compositions were prepared as set forth in Examples 3 and 4.

The high viscosities of the compositions substantially slows the particle sedimentation rate to an extent that no resuspension processing is necessary or required over the estimated shelf life, e.g., about 2 years, of the compositions. These compositions can be marketed in prefilled syringes since they can not easily be removed by a needle and syringe from a container. However, with the compositions in prefilled syringes, the compositions can be effectively injected into the posterior segment of an eye of a human using a 27 gauge or a 30 gauge needle to provide a desired therapeutic effect in the human eye.

The sodium hyaluronate powders used in these compositions (as well as in the other compositions identified in the Examples herein) have water contents in a range of about 4% to about 20%, preferably about 4% to about 8%, by weight. Differences in the average molecular weight of the hyaluronate used can result in variation in the viscosity of compositions in accordance with the present invention which have the same nominal chemical make-ups. Thus, the viscosities indicated herein should be understood to be target viscosities, with the composition preferably having an actual viscosity which is about ±30-35% of the target viscosity, and more preferably ±25% of the target viscosity.

Because each of the compositions set forth in the Examples has a density of about 1 gm/ml, the percentages set forth herein as being based on weight per volume (w/v) can also be considered as being based on weight per weight (w/w).

The compositions of Examples 1 and 2 use or contain a sufficient concentration of high molecular weight (i.e. polymeric) sodium hyaluronate so as to form a gelatinous plug or drug depot upon intravitreal injection into a human eye. Preferably the average molecular weight of the hyaluronate used is less than about 2 million, and more preferably the average molecular weight of the hyaluronate used is between about 1.3 million and 1.6 million. The triamcinolone acetonide particles are, in effect, trapped or held within this viscous plug of hyaluronate, so that undesirable pluming does not occur upon intravitreal injection of the formulation. Thus, the risk of drug particles disadvantageously settling directly on the retinal tissue is substantially reduced, for example, relative to using a composition with a water-like viscosity, such as Kenalog® 40. Since sodium hyaluronate solutions are subject to dramatic shear thinning, these formulations are easily injected through 27 gauge or even 30 gauge needles.

The most preferred viscosity range for the Example 1 and 2 formulations is 140,000 cps to 280,000 cps at a shear rate 0.1/second at 25° C.

The triamcinolone acetonide used in the formulations set forth herein has the chemical name 9-Fluoro-11,21-dihydroxy-16,17-[1-methylethylidenebis(oxy)]pregna-1,4-diene-3,20-dione, and can have the following structure:

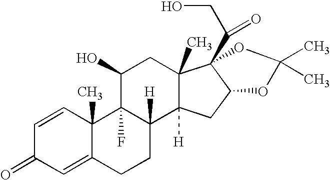

The molecular formula of triamcinolone acetonide is C₂₄H₃₁FO₆ and its molecular weight is 434.49. Triamcinolone acetonide has a very low solubility in water of only about 0.11 mg/ml to about 0.13 mg/m L. Loftsson T. et al., Determination of Aqueous Solubility by Heating and Equilibration: A Technical Note, AAPS Pharm Sci Tech. 2006;7(1): Article 4.DOI: 10.1208/pt070104, and; Yang J. et al., Transdermal delivery system of triamcinolone acetonide from a gel using phonophoresis, Arch Pharm Res 29(5); 412-417: 2006.

The Examples 1 and 2 formulations are prepared as sterile products of a uniform, opaque white dispersion of microfine triamcinolone acetonide particles suspended in a hyaluronate-based polymeric hydrogel, intended for intravitreal injection.

The Examples 1 and 2 formulations can be used top treat, for example, macular edema in patients with diabetes, central retinal vein occlusion, and branch retinal vein occlusion. Notably the Examples 1 and 2 formulations are formulated using only excipients that are fully compatible (i.e. non-toxic) to the eye, particularly to the retina. The Examples 1 and 2 formulations (2% (w/w) and 8% (w/w) triamcinolone acetonide, respectively) are buffered at physiological pH with a low concentration of sodium phosphate salts; rendered isotonic with sodium chloride, and use Water for Injection, USP, as the vehicle.

A target dosage of 1 mg of the triamcinolone acetonide active agent can be delivered in a 50 mg (approximately 48 μL) injection of the Example 1 2% (w/w) triamcinolone acetonide gel suspension formulation. A target dosage of 4 mg of the triamcinolone acetonide active agent can be delivered in a 50 mg (approximately 48 μL) injection of the Example 2 8% (w/w) triamcinolone acetonide gel suspension formulations.

As noted, the triamcinolone present in our formulations does not rapidly, or even slowly, settle out from or precipitate from the formulations. Significantly our Example 1 and 2 formulations have a shelf life of at least two years, meaning that these formulations can be left standing (without agitation) for up to about two years before administration to an eye, and after two years the same formulations can still provide a consistent and accurate dose of triamcinolone upon injection to the formulation to an eye. For example, upon preparation (as set forth in Example 3), 50 μL of our 8% formulation provides 4 mg of triamcinolone acetonide, and if left standing for up to about 2 years 50 μL of our 8% formulation stills provides 4 mg±15% of triamcinolone acetonide, thereby meeting the U.S.P. definition of consistent dosage after storage.

As noted, the composition of triamcinolone 2% injectable gel suspension (Example 1) is triamcinolone 2.0% (w/w), sodium hyaluronate, sodium chloride, dibasic sodium phosphate (heptahydrate), monobasic sodium phosphate (monohydrate), and water for injection). The composition of triamcinolone 8% injectable gel suspension (Example 2) is triamcinolone 8.0% (w/w), sodium hyaluronate, sodium chloride, dibasic sodium phosphate (heptahydrate), monobasic sodium phosphate (monohydrate), and water for injection.

The triamcinolone acetonide injectable gel suspension we have invented is a viscous suspension of triamcinolone acetonide formulated at concentrations of 2% and 8% with sodium hyaluronate, sodium chloride, dibasic sodium phosphate (heptahydrate), monobasic sodium phosphate (monohydrate), and water for injection (i.e. the formulations of Examples 1 and 2 respectively). The suspensions are prepared to have physiologic pH, and to be isotonic, and preservative-free. The Examples 8 and 9 suspensions can be supplied in single-use glass syringes with fixed 27 gauge needles. The syringes are overfilled to 0.17-0.18 mL, and calibrated to deliver 0.05 mL when primed to a black or blue mark on the barrel of the syringe to thereby provide the 2% and 8% suspensions to deliver 1 mg and 4 mg of triamcinolone, respectively (the pre-filled syringes are made by Allergan, Inc., Irvine, Calif.). These syringes have a shelf life of at least about two years when stored at 2-8° C.

### Example 3

### Method for Making Injectable Triamcinolone Acetonide Gel Suspension Formulations

Preferred methods were developed for making the formulations of Examples 1 and 2. The triamcinolone formulations are made as sterile, uniform, opaque white gel suspensions suitable for intraocular (such as intravitreal) injection. The manufacturing process involves two main stages: 1) sterile suspension bulk compounding and 2) aseptic filling. The bulk product manufacture includes preparations of three separate parts, followed by aseptic combination of these three parts. The aseptic filling operation is conducted in a class 100 environment, and the sterile bulk product may be filled into pre-sterilized ready-to-use syringes.

Micronized triamcinolone acetonide, USP, was purchased from Pfizer, Inc., Kalamazoo Mich. Typical and most useful particle sizes for this drug are 4-8 microns in diameter. Sodium hyaluronate gel was purchased from Hyaluron, Woburn, Mass. Typical and most useful molecular weights for this polymer are 1.0 to 1.9 million Daltons. When used, SBE7-β-cyclodextrin (Captisol®) was obtained from CyDex, Inc., Overland Park, Kan.

Part I is prepared in a main batch vessel that has capabilities of bulk heat sterilization and viscous fluid mixing. First, water for injection (WFI) at 40% of batch size is charged into the vessel and sodium chloride is dissolved. Triamcinolone powder is then added and dispersed with strong agitation. In the main batch vessel the suspension is heated and sterilized at above 121° C. for a sufficient time period by steam passing through the jacket of the vessel. After the bulk heat cycle is completed, the suspension is cooled down to room temperature.

Part II is prepared in an open vessel equipped with a top entering, variable speed mixer. First, WFI at 10% of batch size is charged into the vessel. Sodium phosphate salts and, optionally, a β-cyclodextrin derivative is added and dissolved. If necessary, the pH of the solution is adjusted with 1 N sodium hydroxide and/or 1 N hydrochloric acid. When a beta cyclodextrin is used in the formulation is can be dissolved along with the phosphate salts in this part II.

Part III is prepared in a Class 100 environment through a series of aseptic procedures. First, sodium hyaluronate is dissolved in WFI at dilute concentration, e.g., 0.2% w/w. The solution is sterile-filtered and sodium hyaluronate powder is recovered through bulk lyophilization. Finally, the sodium hyaluronate powder is reconstituted with sterile WFI at 50% of batch size.

Sterile bulk suspension is compounded by aseptically combining (mixing) the three parts. First, Part II solution is filtered into sterile Part I already present in the main batch vessel using a 0.2 micron sterilizing grade filter. Part III is then aseptically transferred into the main batch vessel. Finally, the bulk is blended (mixed) under low shear conditions to achieve uniformity. The final bulk suspension is held in a controlled area before aseptic filling.

Aseptic filling operations are performed in a Class 100 environment. Sterile bulk suspension is first filtered through a clarification screen into a sterile holding container. The bulk is then transferred to the filling machine and filled into pre-sterilized syringes. The filled units are transferred to the packaging area for application of tamper-evident seals, labeling and cartoning.

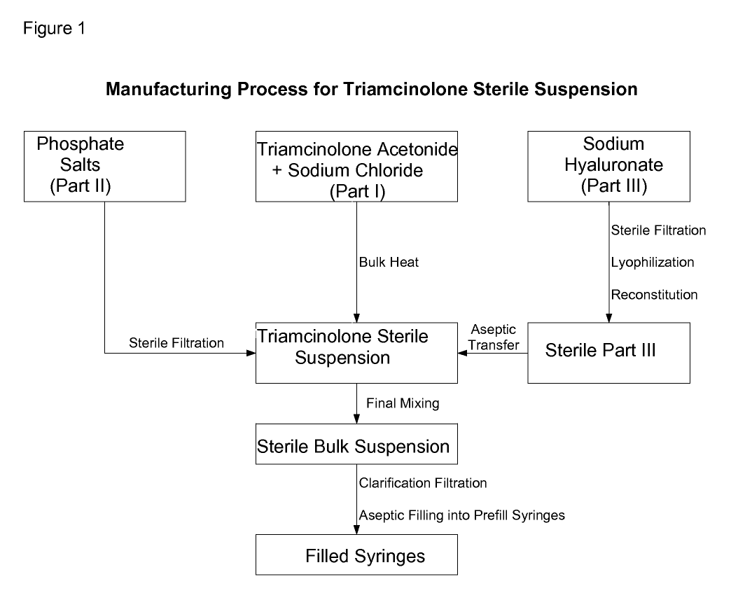

The pharmaceutical manufacturing process of this Example 10 for making triamcinolone sterile suspensions is illustrated by the FIG. 1 process flow chart.

Although not shown in FIG. 1, after Part III has been made (and before the lyophilization step is applied to Part III), Part III can be heated at between about 120° C. and about 130° C. for between about 25-35 minutes. Doing so both sterilizes the hyaluronate and can reduce the initial 1 million to 1.9 million Daltons molecular weight of the hyaluronate used in our formulation by about 20% to about 30% (i.e. to between about 0.7 million to about 1.3 million Daltons), thereby permitting use of a higher (i.e. 30 gauge) gauge injection needle.

### Example 4

### Improved Process for Making Injectable Triamcinolone Acetonide Gel Suspension Formulations

An improved process was developed for making the formulations of Examples 1 and 2. This process was used to make the 8% Trivaris pharmaceutical composition administered to treat a human disease or condition. The same process can be used to make other Trivaris formulations, such as 2% and 4% Trivaris. The disease condition treated can be an ocular disease or condition, such as macula edema (i.e. diabetic macula edema) or a non-ocular condition, such as an articular pathology (i.e. arthritis).

The bulk product compounding process set forth in this Example uses two major processing vessels. One vessel is used for bulk heat sterilization of triamcinolone acetonide aqueous suspension and the suspension is then quantitatively transferred into a second vessel (the main batch vessel) for final mixing with sterile sodium phosphate buffer and sterile sodium hyaluronate powder to sterile gel suspension bulk product. It was determined that triamcinolone acetonide aqueous suspension with sodium chloride is stable during bulk heat sterilization at about 122-128 C. for about 60-90 minutes with no measurable amount of the principal degradation product, triamcinolone, or any unspecified degradation products.

An alternate compounding process can use one processing vessel for bulk heat sterilization of triamcinolone acetonide aqueous suspension and final bulk mixing, as in Example 3.

This process can be referred to as TRIVARIS™ (triamcinolone acetonide injectable suspension, USP) bulk drug product compounding process. A detailed manufacturing process was developed based upon the desired unique characteristics of the final drug product. These characteristics are firstly that the drug product must be sterile so that it is suitable to administer to humans. Secondly, the drug product must be a gel suspension. Thus, although the active pharmaceutical ingredient present (“API”) in the drug product can be one or more of a variety of different therapeutic agents (and in TRIVARIS™ the API is triamcinolone acetonide), the API is suspended in the gel, that is little or none of the API is in solution in the gel. The gel provides a low immunogenicity carrier and sustained release vehicle for the API, as set forth previously. Additionally, the gel used in the formulation is a high viscosity, high shear rate gel which can be administered through a 27 to 30 gauge syringe needle, which is important for patient comfort, ease of injection and rapid self-healing at the injection site. Thirdly, the drug product is packaged as a pre-filled syringe ready for injection. Presentation as a ready to use syringe assists maintenance of sterility and removes the need for any formulation or dose preparation steps prior to injection.

Because the drug product comprises a labile small molecule API, and carrier gel the final drug product cannot be terminally sterilized. Thus, use of either terminal heat or gamma irradiation is contraindicated because either of these common sterilizations methods will cause significant degradation of the carrier gel (i.e. the polymeric sodium hyaluronate) and some degradation of the API. Additionally, not only is terminal sterilization not useable but as well sterile filtration of all parts (constituent ingredients) of the drug product is precluded because the desired drug product is a gel suspension which cannot be filtered because the gel will not pass through the small pore size of a sterile filter and as well filtering would remove the API which is suspended in the drug product as drug particles (i.e. triamcinolone acetonide crystals).

Due to the desired characteristics of the final drug product and the constraints for maintaining sterility of the desired drug product a two stage manufacturing process was developed. In the first stage a sterile gel suspension bulk drug product was compounded. In the second stage the drug product resulting from the first stage is aseptically filled into pre-sterilized syringes and final packaging occurs.

The critical and novel features of the manufacturing process developed are the operations used in first stage, that is, the sterile bulk drug product compounding.

A fundamental aspect of the first stage (sterile bulk drug product compounding) of the manufacturing process developed was to prepare the desired drug product using three parts. The three parts are three in-process sterile parts which are prepared and then mixed together sequentially, one after the other, into a sterile main batch vessel to thereby obtain the final sterile uniform consistency polymeric sodium hyaluronate gel in which the triamcinolone acetonide crystals are uniformly suspended.

The three parts made during the first stage are part 1, a triamcinolone acetonide aqueous suspension with sodium chloride, part 2, sodium phosphate buffer, and part 3 a sterile sodium hyaluronate gel.

Part 1, Triamcinolone Acetonide Aqueous Suspension

To make part 1, a triamcinolone acetonide aqueous suspension in sodium chloride solution was prepared in a jacketed vessel (the first vessel) equipped with a dissolver mixer and a scraper mixer. First, sodium chloride is dissolved in water for injection (WFI) in a separate stainless steel container and transferred into the jacketed bulk heat vessel through a 0.2 micron filter to remove insoluble particulate matter. Triamcinolone acetonide powder was then added and suspended in the solution at a concentration of 20% w/w (the drug concentration in part 1 can be in the range of 115-315% of the drug concentration in the final bulk product, at the end of stage 1). The suspension was then sterilized by bulk heating at a temperature between about 115° C. and about 135° C. for between about 10 minutes and about 90 minutes. More preferably the part 1 sterilization is carried out at a temperature between about 120° C. and about 130° C. for between about 15 minutes and about 60 minutes. Most preferably the part 1 sterilization is carried out at a temperature between about 121° C. and about 128° C. for about 40 to 45 minutes. Part 1 sterilization was carried out using steam (or heating fluid) in the jacket of the vessel with continuous mixing of part 1 in the vessel. The sterilized suspension (part 1) was then cooled to room temperature (25° C.). Part 1 was then ready for sterile transfer into a main batch vessel (the second vessel) through a transfer line with a diaphragm valve.

Triamcinolone acetonide is suspended in an aqueous solution of sodium chloride to form Part 1. Sodium chloride reduces the aqueous solubility of triamcinolone acetonide during the bulk heat sterilization cycle, thereby minimizing the formation of degradants. However, we determined that the presence of phosphate salts during heat sterilization of Part 1 accelerates drug degradation. For this reason, phosphate salts are dissolved separately to form Part 2.

Part 2, Phosphate Buffer

To make part 2, monobasic sodium phosphate and dibasic sodium phosphate was dissolved in WFI in a container and can be prepared at about five to about fifteen times concentration relative to what the buffer concentration is in the final bulk product. More preferably the phosphate concentration in part 2 is about ten times it's concentration is the final bulk product. The buffer solution container was then connected to a pre-sterilized 0.2 micron sterilizing grade filter with a diaphragm valve connected to the main batch vessel to permit sterile filtration of the buffer solution into the main batch vessel.

Part 3, Sterile Sodium Hyaluronate

To make part 3, sterile sodium hyaluronate was obtained from Genzyme Corporation, Cambridge, Mass. in powder form or as a pre-hydrated gel form at a higher concentration (4-6% w/w) than in the final product. As improvement in this process is to not lyophilize or reconstitute the sodium hyaluronate, thereby removing two processing steps. It is preferred to use the sodium hyaluronate powder, as opposed to the pre-hydrated gel, because the powder is easier to reproducibly transfer from its sterile container into the final mixing vessel. The sterile sodium hyaluronate was aseptically packaged into a pre-sterilized sterile transfer assembly which included a container, a connecting tube, and a diaphragm valve. The part 3 container can be a chemically compatible plastic bag or a glass bottle for the powder form, or a stainless steel pressure vessel for the pre-hydrated gel. The transfer assembly was connected to the main batch vessel for sterile transfer, and positive pressure was maintained within the main batch vessel when the prepared sodium hyaluronate was transferred to the main vessel.

When part 3 is added as a pre-formed gel, part 2 and 3 are added separately after the addition of part 1. Thus, part 2 is sterile filtered into the main vessel and part 3 is sterile transferred into the main vessel. The part 3 gel is very viscous and therefore cannot be sterile filtered, even if parts 2 and 3 are mixed together. Part 3, as a pre-formed gel, is instead sterile transferred to the main vessel by application of pressure.

When part 3 is added as a powder, it is preferable to perform the part 3 addition step in the presence of part 2 buffer, as this allows the sodium hyaluronate particles to hydrate into a gel while in direct contact with a uniform mixture of the part 2 components, and at a lower concentration of sodium hyaluronate than would result if the part 2 solution were not present at this process step. The reduced viscosity of the mixture of parts 2 and 3 allows aids the gel particle hydration and mixing.

Final Sterile Gel Suspension Bulk Product

Before transfer of any of the three in-process parts, the main batch vessel, a stainless steel pressure vessel equipped with variable speed ribbon mixer, and the transfer lines was sterilized by steam-in-place (SIP). Sterile connection was made between the three parts and the main batch vessel thereby creating an entirely closed sterile system. The three parts were then sequentially sterile transferred into the main batch vessel. The order of transfer and the final mixing time depends on the form of is sterile sodium hyaluronate (gel or powder) used.

If sterile sodium hyaluronate powder is used, the three in-process parts were transferred in the following order: (1) phosphate buffer (part 2) was sterile filtered into the main batch vessel, (2) sterile sodium hyaluronate powder (part 3) was added into the main vessel by gravity through the diaphragm valve, and (3) a specified quantity of triamcinolone acetonide aqueous suspension (part 1) was sterile transferred into the main batch vessel by pressure while the suspension is mixed in the bulk heat vessel to avoid a concentration gradient. Finally, the three parts were mixed in the main batch vessel at a low speed (about 30 rpm) for between about 66 to 90 hours to achieve a uniform gel suspension.

If pre-hydrated sterile sodium hyaluronate gel is used, the three in-process parts were transferred in the following order: (1) a specified quantity of triamcinolone acetonide aqueous suspension (part 1) was sterile transferred into the main batch vessel by pressure while the suspension is mixed in the bulk heat vessel to avoid a concentration gradient, (2) phosphate buffer (part 2) was sterile filtered into the main batch vessel, and (3) sterile sodium hyaluronate gel (part 3) was transferred into the main vessel by pressurizing the container. Finally, the three parts were mixed in the main batch vessel at a low speed (about 30 rpm) for between about 16 to 24 hours to achieve a uniform gel suspension.

At the end of final mixing, the gel suspension bulk drug product was sterile transferred from the main batch vessel into a sterile bulk product holding vessel by pressurizing the main batch vessel. The bulk product holding vessel was pre-sterilized and connected to the bottom valve of the main batch vessel. The connection was sterilized by SIP together with the main batch vessel prior to the transfer of the three in-process parts and final mixing. The sterile bulk drug product in the holding vessel was then moved into the aseptic filling area for aseptic filling and final product packaging.

There are several advantages to the improved process we developed. Use of a two vessel process permits part 1 to be heat sterilized separately from parts 2 and 3, followed by sequential addition of the three parts to the second vessel. Separate heat sterilization of part 1 is a critical step in the drug product compounding. Another important aspect of the improved process is use of three parts for making the drug product. Thus, as explained above, it was determined that parts 1 and 2 cannot be combined (phosphate induced instability), parts 2 and 3 cannot be combined (the gel is too viscous and sterility would be lost), and that parts 1 and 3 cannot be combined (the hyaluronate would deteriorate upon heating). Hence, the manufacturing process developed maintains a fully sterile formulation (includes a bulk heating step of part 1) and requires that three parts of the drug product be separately prepared and then sequentially combined.

A further advantage of our process over the Example 3 process exists as follows. In the former process the vessel in which part 1 is mixed has two mixers, a low rpm (about 50 rpm) ribbon mixer which creates a low sheer and is for mixing through most of the volume of vessel, and a high rpm (about 10,000 rpm) high sheer homogenous mixer for mixing near the bottom of the vessel. It was found that use of these two mixers in the same part 1 vessel can result in problems such as foaming on the sides (inner walls) of the vessel (which results in loss of material) and lack of uniformity when the solution is discharged from the vessel. Thus, with the current, improved two vessel process, the part 1 vessel has a dissolver mixer with about 300-1000 rpm and a scrapper mixer at 30-50 rpm. And the second or main vessel has only a ribbon mixer. Using two vessels as in this Example thereby permits better mixing of the formulation.

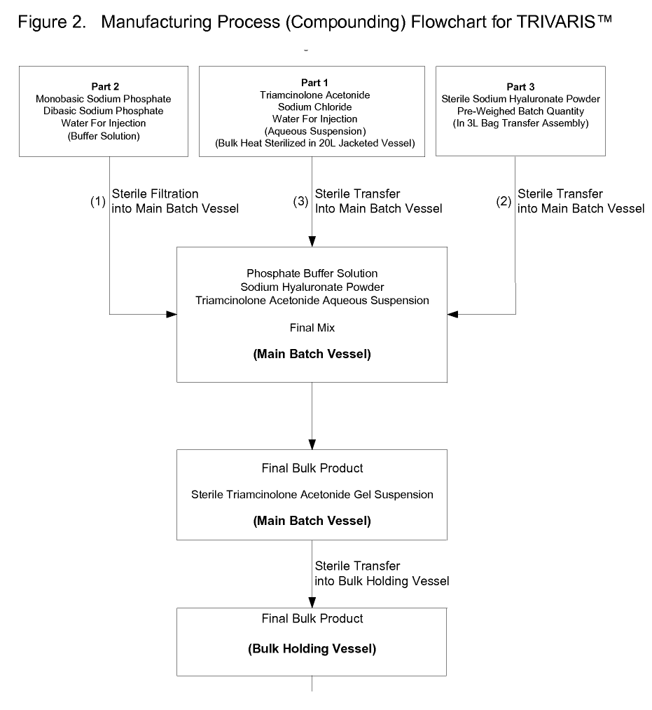

A schematic summary of the improved bulk drug product compounding process set forth in this Example is shown in FIG. 2.

Table 2 sets forth the manufacturing process (amounts and equipment used) to is prepare a four kilogram batch of the final drug product (Trivaris). In Table 2, items 1-7 are compounding steps (first stage) and item 8 is the filling into the syringes step (second stage). Table 3 sets forth the major equipment and components used to carry out a two vessel, three part process within the scope of this Example 4.

TABLE 2
Manufacturing Process Parameters

| Parameter | Value |
|---|---|
| 1. Batch size | 4.0 kg |
| 2. Main batch vessel | Ross 7 liter capacity, 316 L stainless steel pressure |
| | vessel, equipped with a variable speed ribbon mixer |
| 3. Part 1, triamcinolone acetonide aqueous suspension | Bulk heated in Fryma VMF-20, 20 liter capacity, 316 L |
| | stainless steel jacketed pressure vessel, equipped with a |
| | dissolver mixer and a scraper mixer |
| 4. Part 2, sodium phosphate buffer | 400 g, prepared in a stainless steel container |
| 5. Part 3, sodium hyaluronate gel | 5% (w/w) pre-hydrated gel, supplied in a 3 liter stainless |
| | steel pressure vessel by Hyaluron, Inc. |
| 6. Order of transfer to the main vessel^{c} | (1) Triamcinolone acetonide aqueous suspension^{a} |
| | (2) Sodium phosphate buffer |
| | (3) Sodium hyaluronate gel^{b} |
| 7. Final mixing | 30 rpm, 16.5 hours |
| 8. Filling | Inova SV 122 Semi-Automatic Syringe Filler Machine, |
| | capable for low volume and viscous fluid fill. |
| | BD Hypak Stopper Placement Unit (SPU) with a |
| | vacuum system. |
| | Fill approximate 0.17 g into 0.5 mL glass syringes, 100 |
| | syringes (10 × 10) in one tray. |

^{a}Aqueous suspension after bulk heat sterilization as part 1. The quantity of part 1 was 1600 grams transferred based on 20% (w/w) of the aqueous suspension.
^{b}About 2.5 kg of the 5% (w/w) gel was supplied in the vessel, the actual amount of part 3 transferred into main batch vessel was 1840 g so as to provide a 2.3%(w/w) sodium hyaluronate concentration in the final bulk drug product.
^{c}In addition to transferring the three parts, water for injection was added to rinse the transfer line to bring the final bulk drug product to the final batch 4 kg weight.

In the final bulk drug product made per Table 2 batch size the concentration of triamcinolone acetonide was 7.2 wt % to 8.8 wt % (that is 90% to 110% of the 8 wt % label strength), with a pH between 6.9 and 7.7 and a viscosity at a shear rate 0.1/second at 25° C. between 140,000 and 300,000 cps (with an average viscosity is between 225,000 and 240,000 cps), as a uniform suspension with no agglomerates larger than 150 pm present. The entire contents of a prefilled syringe with 8% Trivaris can be readily expelled through a 27 g needle.

TABLE 3
Major Equipment and Components for Commercial Manufacture
(Compounding) of Trivaris ™

| Purpose | Equipment Description |
|---|---|
| Part 1, triamcinolone acetonide aqueous suspension bulk | Fryma VME-20, 20 liter capacity, 316 L stainless steel |
| heat sterilization | jacketed pressure vessel, equipped with dissolver mixer |
| | and scraper mixer |
| Part 2, phosphate buffer container | Appropriate-sized 316 L stainless steel container |
| Part 2, sterile filtration of phosphate buffer solution | Pall Supor ® DCF capsule filter, 0.2 micron sterilizing |
| Part 1, clarification filtration of sodium chloride solution | grade, 500 cm² effective filtration area, (Part #: |
| | CHS92SS) |
| Part 3, sterile sodium hyaluronate powder transfer | 3 L plastic bag with Pall Kleenpak Connector, connected |
| | to a diaphragm value |
| Main batch vessel for final bulk mixing | Ross 7 liter capacity, 316 L stainless steel pressure vessel, |
| | equipped with a triple ribbon mixer |
| Sterile bulk product holding vessel for bulk product hold | 4 liter capacity, 316 L stainless steel pressure vessel |
| and transfer to filling manufacturing site | |

To reiterate the bulk drug product compounding for commercial manufacture of TRIVARIS™ (triamcinolone acetonide injectable suspension, USP) is performed on weight basis. The process set forth in this example 4 includes preparation of three separate parts and sequential transfer of each part into a main batch vessel for final mixing to a uniform gel suspension. Table 4 sets forth the specific ingredient quantities required for commercial manufacture of 8 wt % TRIVARIS™.

TABLE 4
Ingredient Quantities for Commercial Manufacture of TRIVARIS ™
(Triamcinolone Acetonide Injectable Suspension, USP)

| | | Conc. | Qty | | Conc. | Qty |
|---|---|---|---|---|---|---|
| | | In Part | In Part | | In Batch | In Batch |
| Part | Ingredient | (w/w) | (gram) | | (w/w) | (gram) |
| Part 1 | Triamcinolone Acetonide, | 20% | 2000 | g^{a} | 8% | 320 g |
| Triamcinolone | USP | | | | | |
| Acetonide Aqueous | Sodium Chloride, | 1.58% | 158 | g | 0.63% | 25.2 g |
| Suspension | USP/Ph Eur | | | | | |
| | Water For Injection, | q.s to 100% | q.s to 10000 | g | N/A | |
| | USP/Ph Eur | | | | | |
| | Quantity Transfer to Main | 1490 | | | g^{b} | |
| | Batch Vessel | | | | | |
| Part 2 | Dibasic Sodium Phosphate | 3.0% | 12.0 | g | 0.3% | 12.0 g |
| Phosphate Buffer | Heptahydrate, USP | | | | | |
| | Monobasic Sodium | 0.4% | 1.60 | g | 0.04% | 1.60 g |
| | Phosphate, Monohydrate, | | | | | |
| | USP | | | | | |

| | | | | |
|---|---|---|---|---|
| Water For Injection, | q.s. to 100% | q.s. to 1200 | g | N/A |
| USP/Ph Eur | | | | |

| | Quantity Transfer to Main | 400 | g^{d} | |
|---|---|---|---|---|
| | Batch Vessel | | | |
| Part 2 | Water For Injection, | 1400 | g | |
| Filter/Line Rinse | USP/Ph Eur, Add to Main | | | |
| | Batch Vessel | | | |
| Part 3 | Sterile Sodium Hyaluronate, | 92.0 g^{c} | 2.3% | 92.0 g |
| Sterile Sodium | Ph Eur | | | |
| Hyaluronate Powder | | | | |

| | | | |
|---|---|---|---|
| Final Rinse and | Water For Injection, | q.s. to 4000 | g |
| Q.S. | USP/Ph Eur, Add to Main | | |
| | Batch vessel | | |

| | | |
|---|---|---|
| Batch Total Weight | 4000 | g |

^{a}Theoretical quantity for 20% w/w triamcinolone acetonide aqueous suspension. Actual weight is corrected based on “as is” assay value.
^{b}Theoretical quantity of 20% w/w triamcinolone acetonide aqueous suspension to be transferred is 1600 g for final concentration at 8%. Actual quantity to be transferred is 1490 g, a 7% adjustment due to water loss during bulk heat sterilization.
^{c}Pre-weighed quantity by supplier, on dry basis corrected for moisture content in powder.
^{d}The remaining 800 g of buffer solution is used for bioburden test.

While this invention has been described with respect to various specific examples and embodiments, it is to be understood that the invention is not limited thereto. For is example, the corticosteroid formulations set forth herein can be used to treat ocular conditions as well as other non-ocular condition including articular pathologies, such as rheumatoid and osteoarthritis, and spinal conditions, such as facet arthritis, and the treatment of chronic pain by epidural or spinal root injections of a formulation such as a TRIVARIS™.

Additionally, although preferably the polymeric hyaluronate used in TRIVARIS™ is a non-cross linked hyaluronate (so as to obtain, upon application of force to the plunger of the syringe used to administer Trivaris, a high shear rate and hence relative ease of injection of Trivaris through a 27-33 gauge needle), the hyaluronate can alternately be a cross linked hyaluronate (to form a true hydrogel therefore) with a significantly lower viscosity (i.e. with a viscosity of about 5,000 cps at a shear rate of about 0.1/second at about 25° C.). Such a cross-linked hyaluronate can have the same or similar excellent corticosteroid suspension property of TRIVARIS™, and have the additional advantage of longer residency (i.e. biodegradable at a slower rate) of the hyaluronate in the vitreous, with resulting prolonged nominal immunogenicity of such a cross-linked hyaluronate formulation in the vitreous, due to a longer period of intravitreal (or intraocular) retention of the corticosteroid particles in the polymeric matrix of the cross-linked hyaluronate.

Furthermore, besides hyaluronate other cross-linked polymers can be used, such as for example a polycarbophil.

All references, articles, publications, patents and applications set forth above are incorporated herein by reference in their entireties.

We claim:

1. A process for making a pharmaceutical composition, the process comprising the steps of:
(a) preparing a first part comprising an aqueous suspension of particles of an anti-inflammatory steroid;
(b) sterilizing the first part;
(c) preparing a second part comprising a sterile buffer solution for providing a physiological pH to the pharmaceutical composition;
(d) preparing a third part comprising a sterile high viscosity carrier for the anti-inflammatory steroid, and;
(e) mixing together the first, second and third parts to thereby make a sterile, physiological pH pharmaceutical composition with a viscosity of between about 130,000 cps and about 300,000 cps at a shear rate of about 0.1/second at about 25° C. suitable for treating an inflammatory condition.

2. The process of claim 1 wherein the first part comprises sodium chloride.

3. The process of claim 1 wherein the anti-inflammatory steroid is a triamcinolone.

4. The process of claim 1, wherein the sterilizing step is carried out by heating the first part.

5. The process of claim 1 wherein the second part comprises an aqueous phosphate buffer.

6. The process of claim 1 wherein the third part comprises sodium hyaluronate

7. The process of claim wherein the mixing step is carried out adding part 3 to part 2, followed by addition of part 1.

8. The process of claim 1 wherein the steps of preparing first part and sterilizing the first part are carried out in a first vessel and the mixing step is carried out in a second vessel.

9. The process of claim 1 wherein the steps of preparing first part and sterilizing the first part and the mixing step are carried out in the same vessel.

10. The process of claim 4 wherein the first part is heated to about 115° C. to about 135° C. for between about 10 minutes and about 90 minutes.

11. A process for making a pharmaceutical composition, the process comprising the steps of:
(a) preparing a first part comprising sodium chloride and an aqueous suspension of particles of triamcinolone;
(b) sterilizing the first part by heating the first part
(c) preparing a second part comprising sterile phosphate buffer solution for providing a physiological pH to the pharmaceutical composition;
(d) preparing a third part comprising sterile high viscosity sodium hyaluronate carrier for the triamcinolone, and;
(e) mixing together the first, second and third parts by adding part 3 to part 2 followed by add of part 1, to thereby provide a sterile, physiological pH pharmaceutical composition with a viscosity of between about 130,000 cps and about 300,000 cps at a shear rate of about 0.1/second at about 25° C., suitable for treating an inflammatory condition and injectable through a 27 gauge needle.

12. The process of claim 11 wherein the steps of preparing first part and sterilizing the first part are carried out in a first vessel and the mixing step is carried out in a second vessel.

13. The process of claim 11 wherein the steps of preparing first part and sterilizing the first part and the mixing step are carried out in the same vessel.
